(19) United States
(12) Patent Application Publication
(10) Pub. No.: US 2016/0081826 A1
(43) Pub. Date: Mar. 24, 2016

(54) IMPLANTABLE MEDICAL DEVICE WITH SHAPE MEMORY POLYMER FILTER LAYER

(71) Applicant: BOSTON SCIENTIFIC SCIMED, INC., Maple Grove, MN (US)

(72) Inventors: Gerald Fredrickson, Westford, MA (US); Kristin Regan, Winchester, MA (US); John O'Gara, Ashland, MA (US); Jessica Litchfield, Iowa City, IA (US); Claude O. Clerc, Marlborough, MA (US); William Bertolino, Framingham, MA (US); James P. Rohl, Prescott, WI (US); David R. Wulfman, Minneapolis, MN (US); Devon N. Arnholt, Shoreview, MN (US)

(21) Appl. No.: 14/861,026

(22) Filed: Sep. 22, 2015

Related U.S. Application Data
(60) Provisional application No. 62/054,211, filed on Sep. 23, 2014.

(51) Int. Cl. A61F 2/91; A61F 2/915; A61F 2/00

(52) U.S. Cl. CPC A61F 2/91; A61F 2/0077; A61F 2/915; A61F 2002/009; A61F 2002/041

(57) ABSTRACT

Implantable medical devices and method for making and using implantable medical devices are disclosed. An example implantable medical device may include a tubular body having a plurality of openings formed therein. A filter layer may be disposed along an outer surface of the tubular body. The filter layer may include a shape memory material. The filter layer may be capable of allowing fluids to pass therethrough and may be resistant to tissue ingrowth.

### CROSS-REFERENCE TO RELATED APPLICATIONS

This application claims priority under 35 U.S.C. §119 to U.S. Provisional Application Ser. No. 62/054,211, filed Sep. 23, 2014, the entirety of which is incorporated herein by reference.

### TECHNICAL FIELD

The present disclosure pertains to medical devices, and methods for manufacturing medical devices. More particularly, the present disclosure pertains to implantable medical devices.

### BACKGROUND

A wide variety of intracorporeal medical devices have been developed for medical use, for example, intravascular use. Some of these devices include guidewires, catheters, stents, and the like. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for medical devices. An example medical device may include an implantable medical device. The implantable medical device comprises:

a tubular body having a plurality of openings formed therein;

a filter layer disposed along an outer surface of the tubular body;

wherein the filter layer includes a shape memory material; and

wherein the filter layer is capable of allowing fluids to pass therethrough and is resistant to tissue ingrowth.

Alternatively or additionally to any of the embodiments above, the tubular body may include a shape memory polymer.

Alternatively or additionally to any of the embodiments above, the filter layer has a plurality of pores formed therein.

Alternatively or additionally to any of the embodiments above, at least some of the pores have a diameter of 75 microns or less, or 50 microns or less, or 1 to 50 microns.

Alternatively or additionally to any of the embodiments above, the pores are formed by adding a salt to the filter layer, disposing the filter layer on the tubular body, and then dissolving the salt.

Alternatively or additionally to any of the embodiments above, the filter layer includes a biodegradable material.

Alternatively or additionally to any of the embodiments above, the tubular body includes a stent.

Alternatively or additionally to any of the embodiments above, the stent has isotropic bending characteristics.

Alternatively or additionally to any of the embodiments above, the filter layer is an electrospun layer disposed on the stent.

Alternatively or additionally to any of the embodiments above, the filter layer includes a thermoplastic polyurethane shape memory polymer.

Alternatively or additionally to any of the embodiments above, the filter layer includes a polyhedral silsesquioxane diol.

Alternatively or additionally to any of the embodiments above, the filter layer includes a polyhedral silsesquioxane diol based polyurethane incorporating a poly(lactic-co-glycolic acid) block.

Alternatively or additionally to any of the embodiments above, the filter layer includes a polyhedral silsesquioxane diol based polyurethane incorporating a polycaprolactone block.

Another example medical device comprises:

a stent having an outer surface;

an electrospun fiber filter layer disposed along the outer surface of the stent;

wherein the electrospun fiber filter layer includes a shape memory material having a plurality of pores formed therein; and

wherein the pores are capable of allowing fluids to pass therethrough and are resistant to tissue ingrowth therein.

Alternatively or additionally to any of the embodiments above, at least some of the pores have a diameter of 1 to 50 microns.

Alternatively or additionally to any of the embodiments above, the pores are formed by adding a salt to the electrospun fiber filter layer, disposing the electrospun fiber filter layer on the stent, and then dissolving the salt.

Alternatively or additionally to any of the embodiments above, the filter layer includes a biodegradable material.

Alternatively or additionally to any of the embodiments above, the filter layer includes a thermoplastic polyurethane shape memory polymer.

Alternatively or additionally to any of the embodiments above, the filter layer includes a polyhedral silsesquioxane diol.

Alternatively or additionally to any of the embodiments above, the filter layer includes a polyhedral silsesquioxane diol based polyurethane.

Alternatively or additionally to any of the embodiments above, the stent includes a silicone coating.

An example method for manufacturing a medical device is disclosed. The method comprises:

electrospinning a shape memory polymer fiber onto a stent to form a filter layer on the stent;

wherein the filter layer has a plurality of pores formed therein; and

wherein the pores are capable of allowing fluids to pass therethrough and are resistant to tissue ingrowth therein.

Alternatively or additionally to any of the embodiments above, the pores are formed by adding a salt to the filter layer, disposing the filter layer on the stent, and then dissolving the salt.

Alternatively or additionally to any of the embodiments above, the filter layer includes a polyhedral silsesquioxane diol.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic overview of the biliary and/or pancreatic tree;

FIG. 2 is a perspective view of an example implantable medical device;

FIG. 3 is a perspective view of an example stent or scaffold;

FIG. 4 is a perspective view of another example medical device;

FIGS. 5-8 illustrate an example method for manufacturing an implantable medical device;

FIG. 9 is a side view of an example medical device;

FIG. 10 is a side view of an example medical device;

FIG. 11 schematically illustrates an example electrospun filter member layer;

FIG. 12 schematically illustrates an example stent coated with a filter member; and

FIG. 13 illustrates a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary. The following detailed description should be read with reference to the drawings in which similar elements in different drawings are numbered the same. The drawings, which are not necessarily to scale, depict illustrative embodiments and are not intended to limit the scope of the invention.

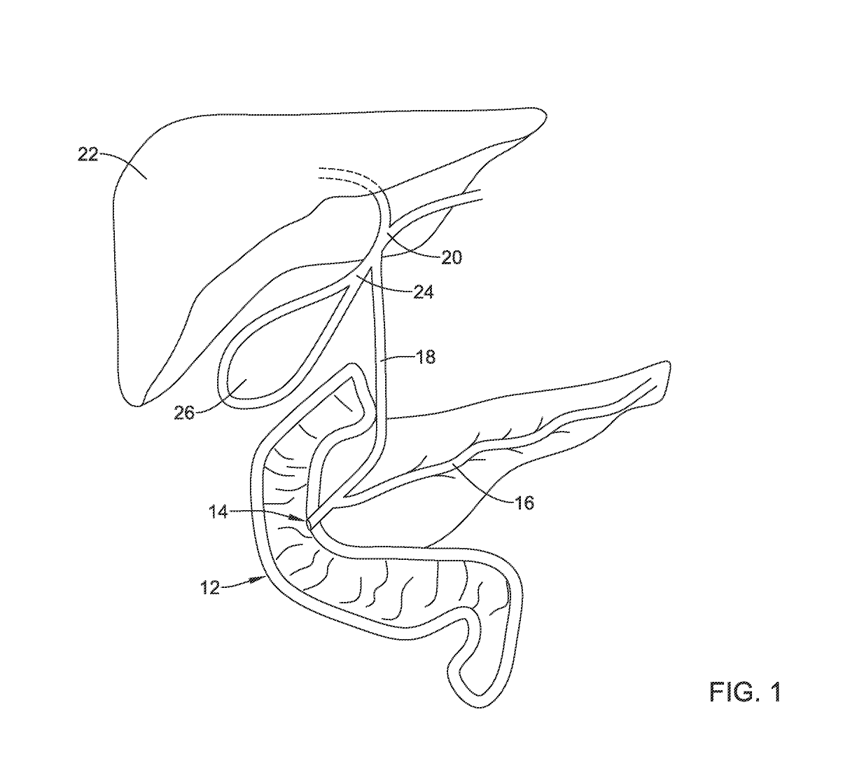

FIG. 1 illustrates an overview of the biliary system or tree. The ampulla of Vater is located at the illustrated portion of the duodenum 12. For the purpose of this disclosure, the ampulla of Vater 14 is understood to be of the same anatomical structure as the papilla of Vater. The ampulla of Vater 14 generally forms the opening where the pancreatic duct 16 and the bile duct 18 can empty into the duodenum 12. The hepatic ducts, denoted by the reference numeral 20, are connected to the liver 22 and empty into the bile duct 18. Similarly, the cystic duct 24, being connected to the gall bladder 26, also empties into the bile duct 18. In general, an endoscopic or biliary procedure may include advancing a medical device to a suitable location along the biliary tree and then performing the appropriate intervention. This may include the implantation of a stent and/or drainage stent.

The present disclosure provides devices and methods for opening strictures and/or providing drainage support (e.g., drainage of fluid, passage of air, etc.) at various target locations along the biliary tree. For example, these devices and methods may allow an implantable medical device, such as a stent, to easily access a particular target location along the biliary and/or pancreatic duct and to drain a fluid from a target location. Furthermore, the devices and methods disclosed herein may be used in other body lumens including, but not limited to, the GI tract (e.g., esophagus, duodenum, bile duct, pancreatic duct, small intestine, large intestine, in a modified anatomy after bariatric surgery, for trans-luminal applications such as pancreatic cyst drainage, or the like), the pulmonary vessels, the airways (e.g., trachea, bronchi, etc.), the vascular system, etc.

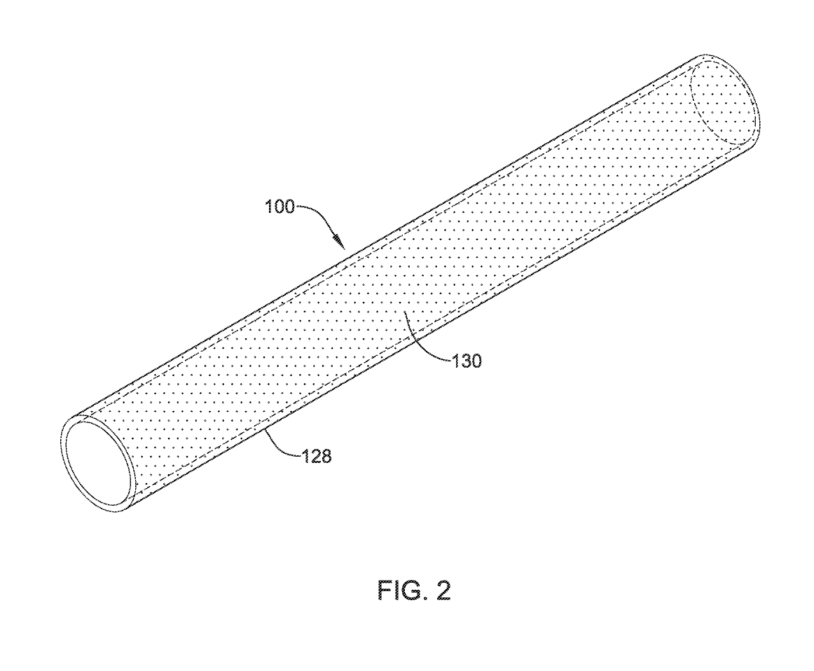

FIG. 2 is a perspective view of an example implantable medical device 100. Device 100 may include a tubular body or filter member 128 having a plurality of openings or pores 130 formed therein. In at least some embodiments, filter member 128 may include a shape memory polymer material. Such a material may allow filter member 128 to be compressed to a reduced profile (e.g., within an endoscope during delivery) and then return to an enlarged profile when delivered. In some instances, filter member 128 may have sufficient radial strength so that filter member 128 may be implanted along a body lumen and the outward radial force of the filter member 128 may secure filter member 128 within the body lumen. In other words, filter member 128 may take the form of a shape memory polymer “stent-like” structure that can be implanted along, for example, the biliary and/or pancreatic tracts in order to open strictures and/or provide drainage support.

Pores 130 may be sized and/or arranged so as to permit the passage of fluids therethrough. In addition, pores 130 may be sized and/or arranged so as to substantially be resistant to tissue ingrowth. In some instances, pores 130 may have a diameter of about 75 microns or less (e.g., 75 microns or less), or about 50 microns or less (e.g., 50 microns or less), or about 1-50 microns (e.g., 1-50 microns). Because of the configuration of filter member 128, implantable medical device 100 may function like a “filter” in that filter member 128 may permit the passage of fluid while restricting the passage of tissue. That being said, filter member 128 may also be termed a drainage member, drainage stent, stent, expandable member, or the like.

The materials used to form filter member 128 may vary. As indicated above, filter member 128 may include a shape memory polymer. The shape memory material may be heat set in an “enlarged” shape or configuration. Because of this, filter member 128 may be compressed into a smaller configuration or shape (e.g., during delivery) and then, upon exposure to the appropriate temperature conditions, revert to the enlarged shape. The enlarged shape may be sufficiently large so that shifting to the enlarged shape exerts a sufficient radially outward force on surround tissue during implantation.

In at least some embodiments, filter member 128 may include a thermoplastic polyurethane shape memory polymer. In some instances, filter member 128 may include a polyhedral silsesquioxane (POSS) diol and/or a POSS-based material. For example, filter member 128 may include a POSS diol based polyurethane incorporating a poly(lactic-co-glycolic acid) block or a POSS diol based polyurethane incorporating a polycaprolactone block. These are just examples. Other materials are contemplated including biostable polymers.

In some of these and in other instances, filter member 128 (and/or stent 132 and/or other structures of device 100) may be formed from a material that is biodegradable. This may allow filter member 128 to degrade over a known time period. Because of this, filter member 128 may be implanted at a target region and then be allowed to degrade over time without the need of a clinician to retrieve or remove filter member. In other words, addition interventions may not be necessary following the implantation of device 100. Some materials are contemplated for filter member 128, stent 132, and/or other components of device 100 that are both biodegradable (and/or bioabsorbable) and shape memory materials (e.g., polymers that are both biodegradable and shape memory).

Filter member 128 may be formed using an electrospinning process. For example, filter member 128 may be formed by electrospinning polymeric fibers onto a substrate. The substrate may be a mandrel, a stent, or the like. Some addition details regarding the substrate and the manufacturing process is disclosed herein.

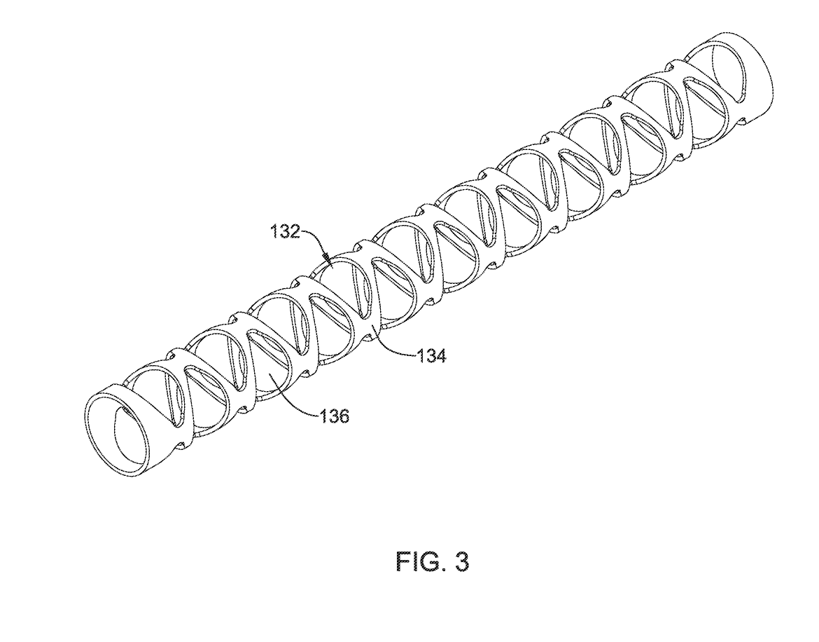

In some instances, filter member 128 may be implanted directly along a target region within a patient. When doing so, the shape memory capabilities of filter member 128 may have a sufficient outward radial force in order to securely implant filter member 128. In other instances, filter member 128 may not be capable of exerting a sufficient outward radial force to securely implant filter member 128 and/or additional structural support may be desired. In these instances, additional structural support may be utilized with filter member 128 in order to increase the radial force. For example, FIG. 3 illustrates an example stent or scaffold 132 that may be used in conjunction with filter member 128 in order to provide additional radial force and/or structural support. Stent 132 may include a stent body 134 having a plurality of openings 136 (e.g., stent openings 136) formed therein. Openings 136 may allow stent 132 to be sufficiently flexible so as to not adversely impact the flexibility of filter member 128. For example, openings 136 may be arranged so that stent 132 has substantially isotropic bending characteristics. In addition, stent 132 may be formed from a shape memory material (e.g., a nickel-titanium alloy such as nitinol) that is set in an enlarged shape so as to increase the radial force exerted during implantation of stent 132 (and/or stent 132 with filter member 128 disposed along the outer surface thereof). In other embodiments, stent 132 may be a balloon expandable stent (e.g., formed from a material that is not a shape memory material).

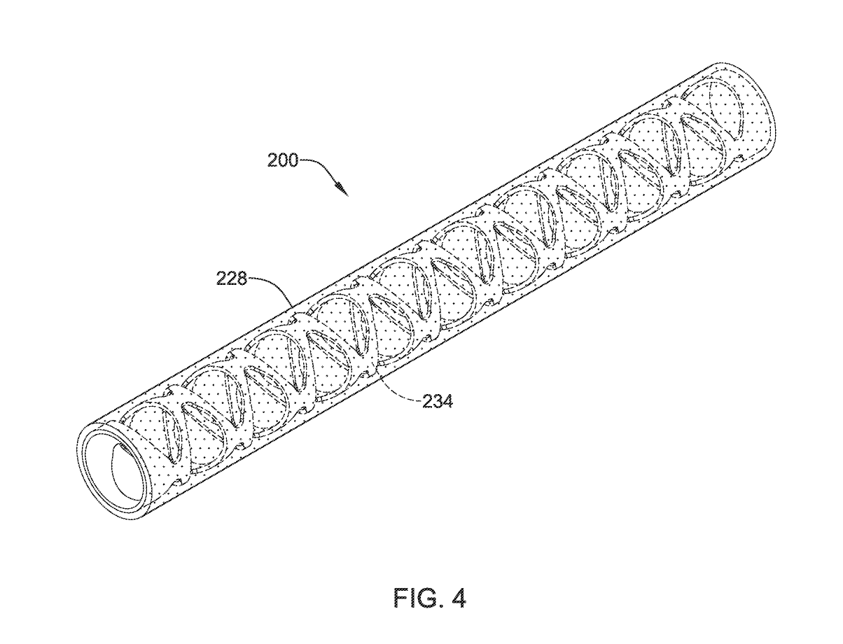

FIG. 4 illustrates another example implantable medical device 200 that may be similar in form and function to other implantable medical devices disclosed herein. Device 200 may include stent 234, which may be the same or similar to stent 132. Filter member or layer 228, which may be the same or similar to filter member 128, may be disposed along stent 234, for example along the outer surface of stent 234. Just like device 100, device 200 may be implanted along a body lumen (e.g., along the biliary and/or pancreatic tract) in order to open strictures and/or provide drainage support. Filter member 228 may extend along essentially the entire length of stent 234 or a portion of the length of stent 234 (e.g., stent 234 is a partially covered stent). Stent 234 may be a self-expanding stent or a balloon expandable stent. Stent 234 may be formed from a suitable material such as a nickel-titanium alloy (e.g., nitinol), cobalt-chromium-molybdenum alloys (e.g., UNS: R30003 such as ELGILOY®, PHYNOX®, and the like), a polymer material (e.g., polyethylene terephthalate, or the like), or the like. Stent 234 may be formed by laser cutting, weaving one or more filaments (e.g., in a braided or knit pattern).

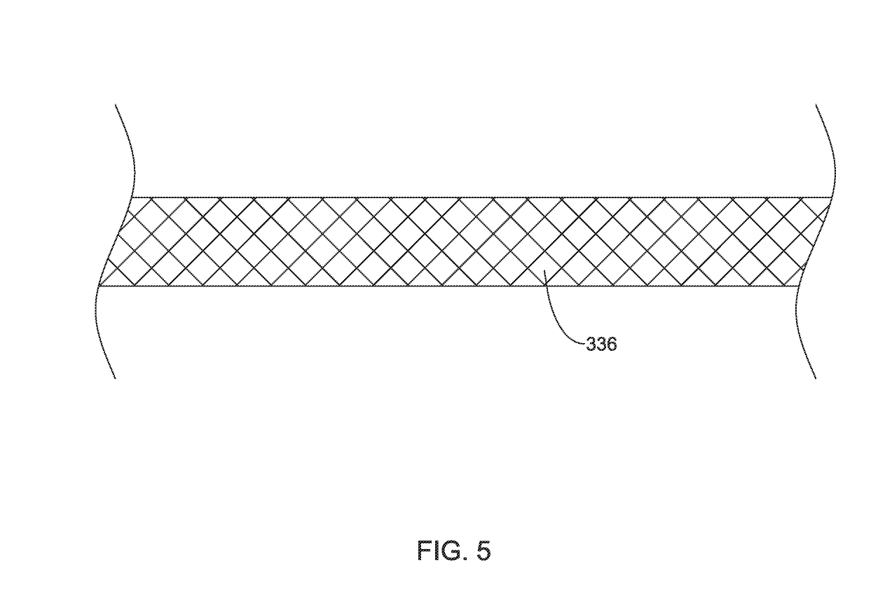

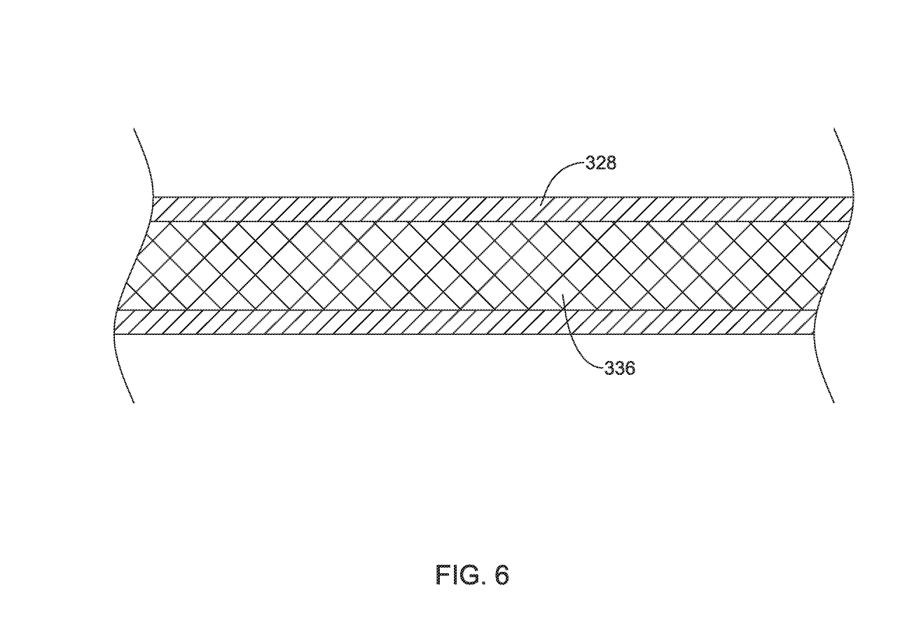

FIGS. 5-8 illustrate an example method for manufacturing a medical device such as any of those disclosed herein. For example, FIG. 5 illustrates an example mandrel 336. In some instances, mandrel 336 may have a smooth outer surface. In other embodiments, mandrel 336 may have an uneven surface so as to help define pores in filter member/layer 128/228. In still other embodiments, mandrel 336 may be stent 132/232. A layer of material 328 may be disposed along mandrel 336 as shown in FIG. 6. Material 328 may ultimately define filter member/layer 128/228. In some instances, material 328 may be fibers that are electrospun onto mandrel 336. In other instances, material 328 (and, ultimately, filter member/layer 128/228) may be formed using a suitable molding process, dipping process, extrusion process, or the like.

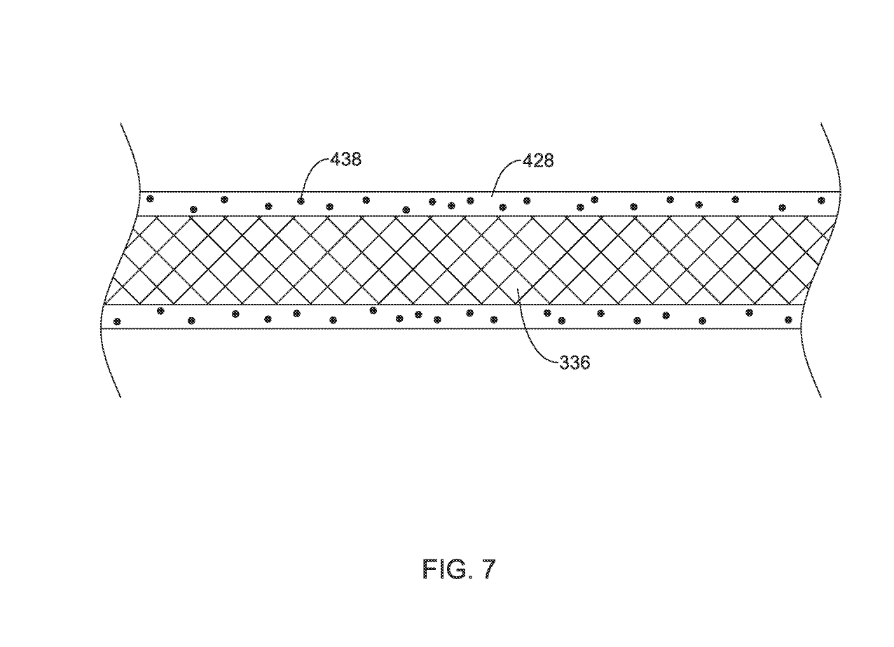

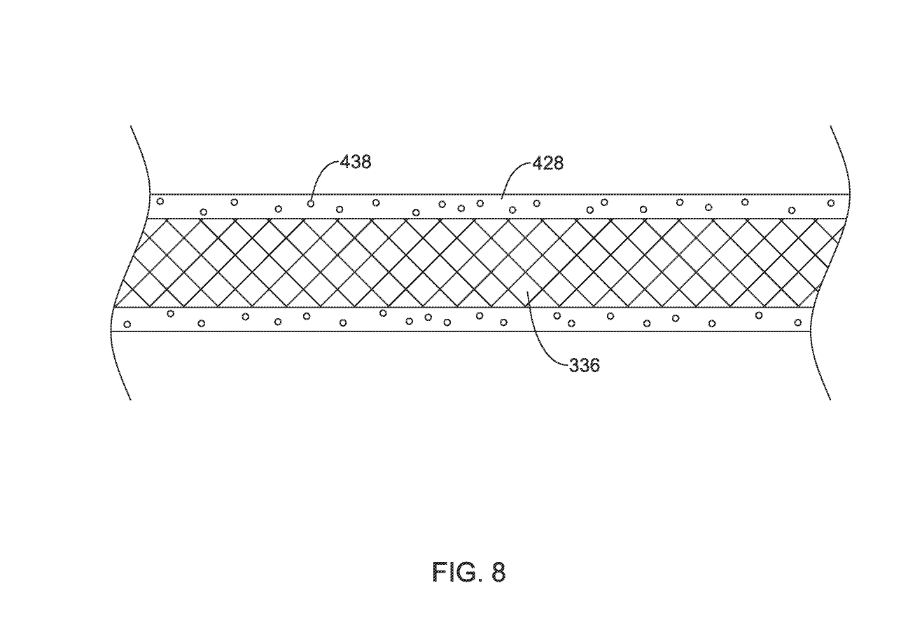

Pores (e.g., similar or the same as pores 130 in filter member 128) may be formed in material 328. This may include mechanically forming the pores. Alternatively, other processes may be utilized. For example, in some instances, a layer of material 428 that includes salt particles 438 may be disposed along mandrel 336 as shown in FIG. 7. Salt particles 438 may be dissolved to define pores 430 as shown in FIG. 8. Alternatively, material 428 may include a miscible polymer that is mixed into material 428 and then subsequently dissolved to define pores 430.

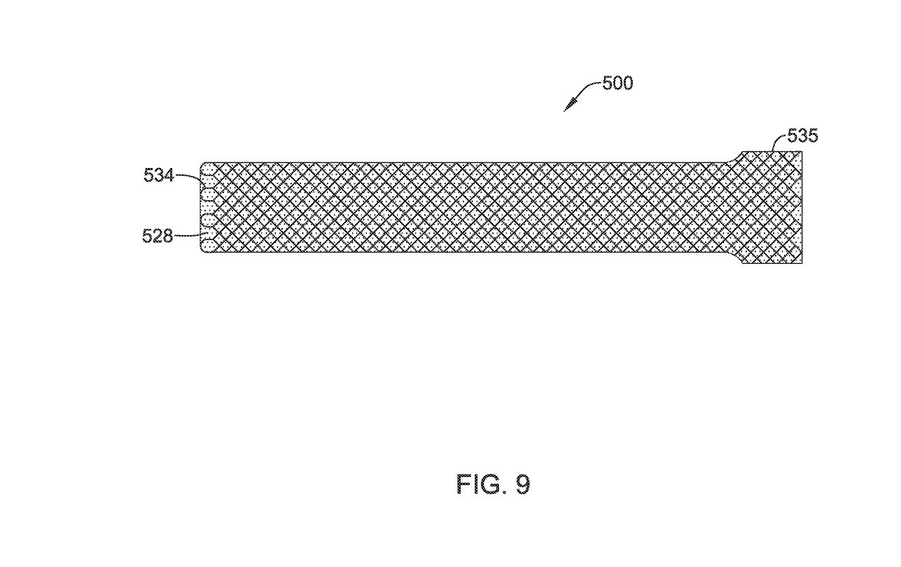

FIG. 9 illustrates an example medical device 500 similar to other devices disclosed herein. Device 500 may include a stent 534 with a filter member 528 disposed thereon. Filter member 528 may be similar in form and function to other filter members disclosed herein (e.g., filter member 528 may include pores that are capable of allowing fluids to pass therethrough and is resistant to tissue ingrowth). In this example, device 500 may take the form of an esophageal stent. Stent 500 may include a widened end region or flare 535. Flare 535 may be disposed at the distal end of stent 500, the proximal end of stent 500, or both. Filter member 528 may cover all or only a portion of stent 500.

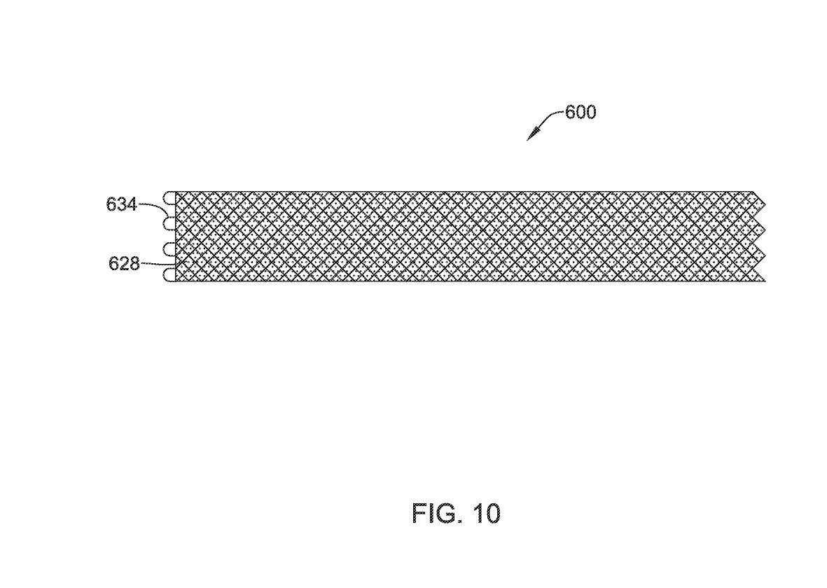

FIG. 10 illustrates an example medical device 600 similar to other devices disclosed herein. Device 600 may include a stent 634 with a filter member 628 disposed thereon. Filter member 628 may be similar in form and function to other filter members disclosed herein (e.g., filter member 628 may include pores that are capable of allowing fluids to pass therethrough and is resistant to tissue ingrowth). In this example, device 600 may take the form of a biliary stent. Filter member 628 may cover all or only a portion of stent 600.

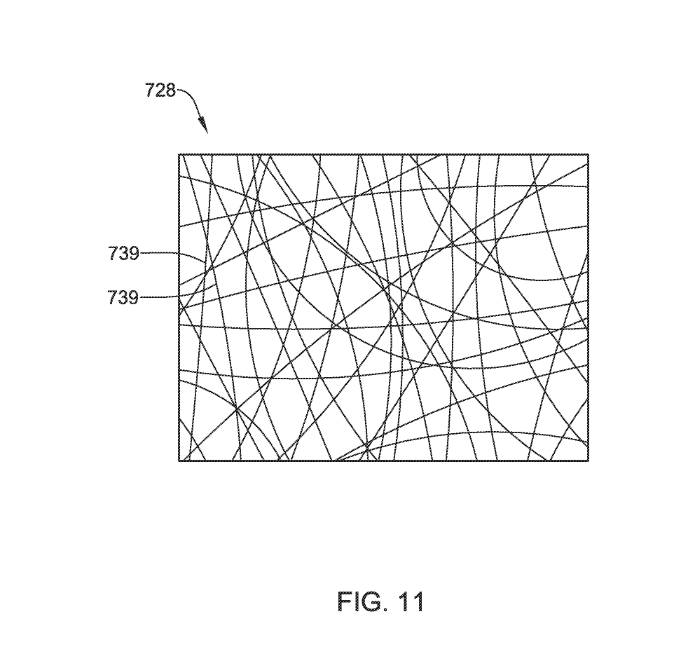

Filter member 528/628 (and/or other filter members disclosed herein) may be formed by an electrospinning process. In general, the electrospinning process may include pumping a polymer solution through a nozzle held at a high electrical potential. The high electrical field forces a droplet of the polymer into a conical shape (which may be referred to as a “Taylor Cone”) at the tip of which is the highest electrical field. Electromotive force overcomes surface tension, inducing a liquid jet that flies toward the nearest target at the lowest electrical potential. As turbulence develops in the jet, the stream may become unstable causing it to take on a whipping motion (instability region). During the time of flight, the solvents in the solution evaporate and the polymer chains associate and entangle with one another, which continually being drawn by electric field generated force. When properly tuned, solidified polymer fibers may deposit onto the lower potential target. The result may be an electrospun filter member layer 728 including a plurality of electrospun fibers 739 as shown in FIG. 11. Fibers 739 may have a diameter on the order of about 100 nm to about 4 microns, or about 200 nm to about 2 microns.

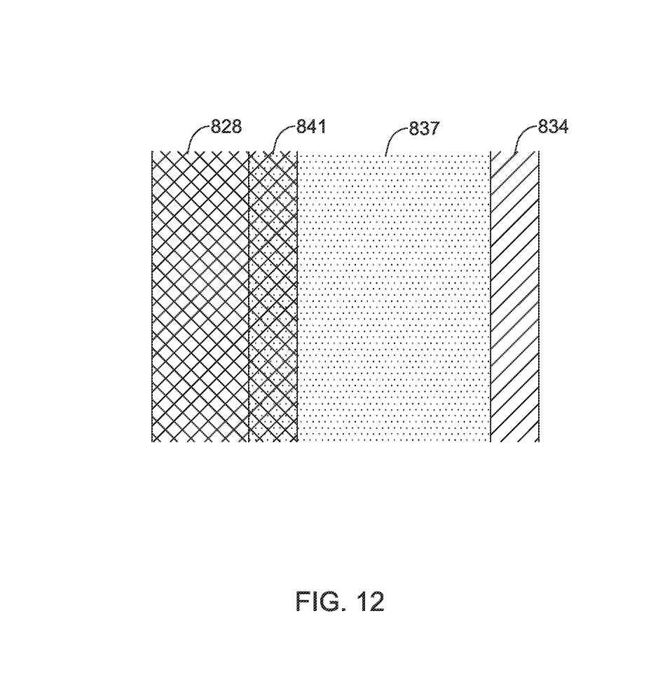

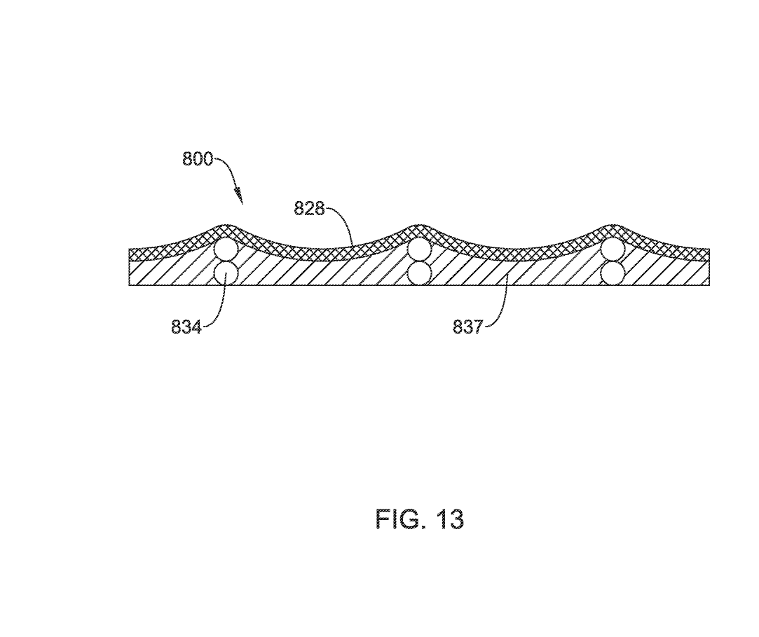

In some instances, it may be desirable to electrospin a filter member layer onto stent 834 as shown in FIGS. 12-13. For example, FIG. 12, which is schematic in nature, depicts that stent 834 may be coated with a coating such as a silicone coating 837. Other coating materials are contemplated including polyurethane, polytetrafluoroethylene (PTFE), expanded polytetrafluoroethylene (ePTFE), or the like. In at least some embodiments, coating 837 may be bioabsorbable. Coating 837 may be integral with stent 834 or may be applied as a by spray coating, dip coating, etc. In some instances, coating 837 may be formed from a sheath or tube that is secured to stent 834. Coating 837 may be disposed along the entire stent 834 or just portions of stent 834.

Filter member 828 may be electrospun onto coating 837. At an interface 841 of filter member 828 and coating 837, coating 837 may surround the fibers of filter member 828 or otherwise the fibers may comingle with coating 837. Coating the fibers of filter member 828 with coating 837 may provide increased strength.

The thickness of filter member 828 may vary. In some instances, filter member 828 may have a thickness on the order of 0.00254 to 0.254 mm (e.g., about 0.0001 to about 0.010 inches) or so. This may include a single layer of fibers or a plurality of layers of fibers stacked on top of each other. In some instances, filter member 828 includes multiple layers of fibers. The layers of fibers may have differing densities. For example, a more radially “outer” layer may have a lower density so as to allow for cell ingrowth and a more radially “inner” layer may have a greater density that prevents cell ingrowth. The “outer” layer may provide a scaffold for cell ingrowth to treat, for example, leaks and fistulas, and allow for easier removal of stent 834 (e.g., after bioabsorption of the “outer” layer). In addition, tissue ingrowth may also aid in securing stent 834 to tissue, which may reduce and/or prevent migration of stent 834. It can be appreciated that in instances where tissue ingrowth is desired, filter member 828 may not be functioning as a “filter” and, instead, may be described as being a covering, coating, or tissue ingrowth promoting member.

In some instances, the fibers of filter member 828 may be disposed only over the struts of stent 834. In addition or in the alternative, the fibers may be disposed between the struts. The fibers of filter member 828 may fully coat stent 834. Alternatively, the fibers may coat only a portion of stent 834. Either of these embodiments may or may not include coating one or more flared ends of stent 834 (e.g., in embodiments where stent 834 includes a flared end similar to flare 535 of stent 500), which may reduce leaking by helping to seal the “top” and “bottom” of stent 834. In some instances, the fibers of filter member 828 may be arranged in a helical pattern about stent 834. In other words, filter member 828 may be helically disposed about stent 834.

The thickness and/or density can also vary along filter member 828. For example, the density and morphology of the electrospun fibers can be varied continuously through the thickness of filter member 828 as well as in different regions of stent 834 by modifying the coating parameters. In some instances, the morphology of the spun fibers can be altered by sintering. Polar solvents can be added to the solution to enhance conductivity. The fiber may also be plasma treated after coating.

In addition to or in the alternative to the materials that are disclosed herein, the electrospun fibers can include materials that are bioabsorbable. A bioabsorbable filter member 828 may provide a scaffold for cell ingrowth to treat, for example, leaks and fistulas, and allow for easier removal of stent 834 after bioabsorption. In some instances, the electrospun fibers can include a pharmaceutical substance (e.g., an antimicrobial). In some of these and in other instances, the electrospun fibers may include a lubricious material, which may help to reduce stent deployment forces. In some of these and in other instances, the electrospun fibers may include hydrophobic materials. In some of these and in other instances, the electrospun fibers may include hydrophilic materials. In some of these and in other instances, the electrospun fibers may include materials that promote cell adhesion. In some of these and in other instances, the electrospun fibers may include a biological material such as collagen.

In some instances, filter member 828 can have several combinations of different layers of fibers and/or materials along the length thereof. This may allow filter member 828 to be tailored to the needs of a particular patient, particular target in the anatomy, etc. In addition, the level of cell ingrowth, fluid transfer, and the like can also be tailored by varying filter member 828.

U.S. Pat. No. 7,067,606 is herein incorporated by reference.

U.S. Pat. No. 7,091,297 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An implantable medical device for use along the biliary and/or pancreatic tree, comprising:
a tubular body having a plurality of openings formed therein;
a filter layer disposed along an outer surface of the tubular body;
wherein the filter layer includes a shape memory material; and
wherein the filter layer is capable of allowing fluids to pass therethrough and is resistant to tissue ingrowth.

2. The implantable medical device of claim 1, wherein the filter layer has a plurality of pores formed therein.

3. The implantable medical device of claim 2, wherein at least some of the pores have a diameter of 1 to 50 microns.

4. The implantable medical device of claim 2, wherein the pores are formed by adding a salt to the filter layer, disposing the filter layer on the tubular body, and then dissolving the salt.

5. The implantable medical device of claim 1, wherein the filter layer includes a biodegradable material.

6. The implantable medical device of claim 1, wherein the tubular body includes a stent.

7. The implantable medical device of claim 6, wherein the stent has isotropic bending characteristics.

8. The implantable medical device of claim 6, wherein the filter layer is an electrospun layer disposed on the stent.

9. The implantable medical device of claim 1, wherein the filter layer includes a thermoplastic polyurethane shape memory polymer.

10. The implantable medical device of claim 1, wherein the filter layer includes a polyhedral silsesquioxane diol.

11. The implantable medical device of claim 1, wherein the filter layer includes a polyhedral silsesquioxane diol based polyurethane incorporating a poly(lactic-co-glycolic acid) block.

12. The implantable medical device of claim 1, wherein the filter layer includes a polyhedral silsesquioxane diol based polyurethane incorporating a polycaprolactone block.

13. An implantable medical device, comprising:
a stent having an outer surface;
an electrospun fiber filter layer disposed along the outer surface of the stent;
wherein the electrospun fiber filter layer has a plurality of pores formed therein; and
wherein the pores are capable of allowing fluids to pass therethrough and are resistant to tissue ingrowth therein.

14. The implantable medical device of claim 13, wherein at least some of the pores have a diameter of 1 to 50 microns.

15. The implantable medical device of claim 13, wherein the pores are formed by adding a salt to the electrospun fiber filter layer, disposing the electrospun fiber filter layer on the stent, and then dissolving the salt.

16. The implantable medical device of claim 13, wherein the filter layer includes a biodegradable material.

17. The implantable medical device of claim 13, wherein the filter layer includes a thermoplastic polyurethane shape memory polymer.

18. The implantable medical device of claim 13, wherein the filter layer includes a polyhedral silsesquioxane diol.

19. The implantable medical device of claim 13, wherein the stent includes a silicone coating.

20. A method for manufacturing a medical device, the method comprising:
electrospinning a shape memory polymer fiber onto a stent to form a filter layer on the stent;
wherein the filter layer has a plurality of pores formed therein; and
wherein the pores are capable of allowing fluids to pass therethrough and are resistant to tissue ingrowth therein.
